(19) United States
(12) Patent Application Publication
(10) Pub. No.: US 2005/0154424 A1
(43) Pub. Date: Jul. 14, 2005

(54) Device for locating the target spot of electrodes used for brain stimulation particularly deep brain stimulation

(76) Inventors: Peter Tass, Dusseldorf (DE); Michael Schiek, Aachen (DE)

Correspondence Address: THE FIRM OF KARL F ROSS, 5676 RIVERDALE AVENUE, PO BOX 900, RIVERDALE (BRONX), NY, 10471-0900, US

(21) Appl. No.: 10/507,957

(22) PCT Filed: Feb. 19, 2003
(86) PCT No.: PCT/DE03/00498

(30) Foreign Application Priority Data
Mar. 14, 2002 (DE) 10211765.9

(51) Int. Cl. A61N 1/18

(52) U.S. Cl. 607/45

(57) ABSTRACT

The invention relates to a device for locating the target spot of electrodes used for brain stimulation, particularly deep brain stimulation. The inventive device comprises an isolation amplifier (1) which is provided with at least one electrode (2) and at least one sensor (3). In a preferred embodiment, said device is provided with a control unit (4) which puts the signals of the electrodes (2) and the sensor (3) in relation and controls the input of stimuli via the electrode. In a particularly preferred embodiment, the stimuli are launched in a galvanically decoupled manner by optical means. The inventive device makes it possible to find the target spot for brain-stimulating electrodes in a particularly precise and fast manner according to objectifiable parameters.

The invention relates to a device for localizing the target point of electrodes or brain stimulation, especially deep brain stimulation.

Symptoms of neurological pathology, like for example akinesis, rigor and tremor are attributable to defects at locally circumscribed regions of the brain. These symptoms can be ameliorated or eliminated for example by deep brain stimulation. Decisive for the stimulation effect, apart from the stimulation parameters, is the location of the stimulation.

In the state of the art, the brain areas are coarsely localized by NMR or computer tomography displays. The precision of these processes is limited by the natural variability of the anatomical structure of the brain and by the limited resolution of the methods based upon physical boundary conditions. Especially the neuron populations responsible for the defect can be small and especially in comparison to the regions localized by the NMR or CT images. It is therefore necessary to undertake a more precise determination of the target point based upon the information oftained by these methods.

In accordance with a known method as has been described for example in the article of Benabid A. L., Pollak P., Gervason C., Hoffmann D., Gao D. M., Hommel M., Perret J. E. De Rougemont J. (1991) Long term suppression of tremor by chronic stimulation of the ventral intermediate thalamic nucleus The Lancet 337, 403-406, for each stimulation location, the expression of the symptom, for example the intensity of the tremor, the influence upon the motor response as well as the presence of rigor and akinesis of a patient is tested by a neurologist. For this purpose an electrode is introduced into the brain which is located at a fixedly predetermined target direction and which can be moved small distances of about 1 mm forwardly and rearwardly. In the respective positions by means of the electrode a stimulation is carried out and the neurologist tests the effects of the stimulation on the symptoms as a function of the excitation neurologically produced at these special points. The patient is thus requested, as a function of the intensity of the stimulation to describe whether he or she finds an improvement or not. The fact that the results depend upon questioning of the patient, however, makes this approach extremely subjective and does not supply any objective parameter.

In another method, which has been described in the publication of Levy R., Hutchison W. D., Lozano A. M., Dostrovsky J. 0., under the title “High frequency synchronization of neuronal activity in the subthalamic nucleus of Parkinsonian patients with limb tremor” of the Journal of Neuroscience 20 (2000) 7766-7775, for each nuclear region respectively of the brain, characteristic discharge patterns in the target region are detected by electrophysiologicalist or an electrophysiologically qualified neurologist by means of microelectrode evaluation. For this purpose characteristic frequencies and/or patterns of the discharge are sought which can be correlated with certain nuclear regions of the brain. With this method however only coarse characteristics are detectable and from them it cannot be readily determined whether or not there is a relationship with the dysfunction. The method does not supply any information as to the functional significance of the detected neuron discharges. The operation is extended by up to three hours.

The known methods of the state of the art are highly time consuming and personnel intensive and give no objective data. They exclusively analyze amplitude related stimulus processes. In addition, by the insertion of the electrodes, small regions of the brain can be damaged and that can result in a reaction upon the response and in spontaneous activity in these regions of the brain which can alter for a certain period of time the results which are obtainable until the corresponding locations heal.

It is therefore the object of the invention to provide a device with which optimum target points in the brain can be rapidly and especially objectively be determined. The discovery of these target points should be facilitated.

Based upon the preamble of claim 1, the objects are attained in accordance with the invention with the features given in the characterizing part of claim 1.

With the device according to the invention it is possible in a rapid manner and way to obtain an objective determination of the optimum target point in the brain. The device enables especially a relationship between the target location in the brain and the dysfunction to be established.

Advantageous features of the invention are given in the dependant claims.

The drawing shows a circuit diagram of the device according to the invention.

The drawing shows

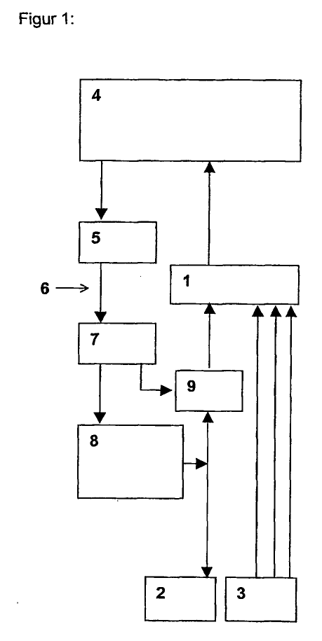

FIG. 1.: a preferred configuration of the device according to the invention in a block diagram.

The device illustrated in FIG. 1 according to the invention encompasses an isolation amplifier (1) connected to at least one electrode (2) as well as sensors (3) for detecting physiological measurement signals. The isolation transformer is also connected with a unit (4) for signal processing and control and which is connected to an optical transmitter (5) for the stimulation. The optical transmitter (5) is connected via a lightwave guide (6) with an optical receiver (7) which is connected with a stimulating unit (8) for signal generation. The stimulating unit (8) for signal generation is connected with the electrode (2). At the input region of the electrode (2) there is a relay (9) or a transistor in the isolation transformer (1).

The device according to the invention can basically operate in three different operational modes:

The electrode (2) measures the neurological activity, for example the local field potential (LFP) whereby
(a) without or
(b) with stimulation of the target region or
(c) alternatingly with and without stimulation of the target region.

The sensors (3) measure an electrophysiological feature which is functionally related to neuronal activity derived from stimulation of the electrode (2) like for example the neuronal activity of another brain region, peripheral tremor or heart frequency, breathing frequency, blood flow, blood pressure, blood gases.

In case (b) the stimulation is effected via the macro or micro electrode (2) which also derives the neuronal activity.

According to the invention, the device allows the discharge in the target region as well as a physiological feature which occurs in combination with the neuronal activity in the target region to be measured. For this purpose, the signals which are delivered by the electrode (2) to the brain are supplied to the isolating amplifier (1) and can be referred to the signals from the sensors (3).

As the sensors (3) for example at least one component from the group of epicordical electrodes, EEG scalp electrodes, deep electrodes, brain electrodes, peripheral electrodes, for example for the measurement of muscular activity or heart frequency, accelerometers or a thermistor for measurement of the breathing frequency can be used. In addition, two or more of the same electrode types can be used.

According to the invention it is thus possible through bivariate or multivariate data analysis, for example, by analysis of the phase synchronization or coherency, to determine a functionality between the neuronal activity at a target point of the brain with an activity at another part of the body or at another location in the brain. With the isolating amplifier (1) it is possible in the simplest case through the use of at least one electrode (2) at whose end a potential difference can be measure to capture a measurement signal from the brain and according to the invention to register that signal with at least one further measurement signal obtained from the sensors (3) and relate the two. For this simple embodiment which falls into the above mentioned class (a) no relay or transistor (9) is necessary.

The electrode (2) can be formed by at least two wires at whose ends a potential difference can be measured or at whose ends a potential difference can be applied. In a further embodiment the electrode (2) can also comprise more than two individual wires which can be provided for detecting a measurement signal or for stimulation of the brain. For example, four wires can be provided in a conductor cable, whereby between different ends a potential difference can be applied or measured. This permits in this manner the magnitude of the investigated or stimulated target region can be varied. The number of wires from which the electrode is composed is limited, with respect to its upper value, only by the resulting thickness of the cable to be introduced into the brain and so that the smallest possible amount of brain material will be damaged. Commercial electrodes encompass four wires but it is also possible to use five, six or more wires or also only three wires.

For the case in which the electrode (2) encompasses more than two wires, at least two of these wires can also function as a sensor (3) so that in this special case an embodiment is provided in which the electrode (2) and the sensor (3) are united in a single component. Apart from this component, sensors (3) which are not structurally united with the electrode (2) can be provided.

In a preferred embodiment of the invention, not only spontaneous signals—thus signals without associated stimulation can be evaluated according to case (a) along with signals which are called forth by a stimulation over the electrodes as in case (b). For this purpose, measurement signals from the electrode (2) are picked up by the isolating amplifier (1) together with signals from the sensors (3) and are supplied to the unit (4) for signal processing and control.

In a further preferred embodiment in case (c), the mode of operation is an alternation between the modes (a) and (b). The device for this purpose encompasses apart from the electrode (2), the isolating amplifier (1) and the sensor (3), a control device (4) for signal processing and control which is connected to the galvanically decoupled transmitter (5) with the stimulating unit (8) for signal generation. In this embodiment the isolating amplifier (1) has a relay or transistor (9) connected ahead of it. With this embodiment the device encompasses means for selectively or preprogramming the switching of the stimulating unit (8).

The unit (4) for signal processing and control encompasses means for a univariate and bivariate data processing to characterize the frequency characteristics and the interaction (for example coherency, phase synchronization and directionality) as has been described for example in “Detection of n:m phase locking from noisy data: application to magnetoencephalography” of P. Tass et al in Physical Review Letters, 81,3291 (1998).

In addition, the signal processing and control unit encompasses preferably means for enabling visualization of the signals and in addition, preferably, for securing [recording] the data. The device according to the invention can, in addition, include a reference data bank which is suitable for identification of brain regions from registered irritation responses and/or identification of spontaneously registered neuronal discharge patterns. The reference data bank can be for example integrated into the controller (4). The signal processing and control can also be effective through various devices 4, 4a (4a not being illustrated in the figure). The processed data is supplied to the optical transmitter (5) for stimulation which is connected to the optical receiver by the lightwave guide (6). Through the optical decoupling of the control signal, the optical receiver in the modes (b) and (c) can be actuated with galvanic decoupling of the stimulation control from the electrode (2). This means that the effect of noise signals is limited. The galvanic decoupling need not use an optical coupling of the control signal and can employ various alternatives approaches. It can for example be effected by an acoustic coupling, operating for example in the ultrasonic range which does not interfere with the investigation. A disturbance-precontrol can also be realized for example through the use or assistance of suitable analog or digital filters. The relay circuit or the transistor ensures that the neuronal activities will be again measured directly following each stimulus without overdriving the isolating amplifier. As the optical receiver (7) a photocell can for example be used. The optical receiver supplies the signal received from the optical transmitter (5) for stimulation to the stimulator unit (8) and the relay (1) [sic (9)]. Through the stimulating unit (8) targeted stimuli are applied by the electrode (2) to the potential target region in the brain. The electrical pulses which are triggered in the brain by the stimulator unit (8) in the targeted region give rise to a reaction which is supplied through the electrode (2) through the relay to the isolating amplifier (1) and which are in the form of brain signals with physiological features which are measured with the sensor (3) and compared to the signal at the brain region supplied by the electrode (2). In this manner a direct functionality can be obtained between the relevant brain region which is explored and the physiological reaction associated therewith.

Through the use of the device according to the invention the following questions can be advantageously explored:
1. Is there a pathological rhythmic activity at the target location? In the case the answer is in the affirmative, the functional significance of this rhythmic activity is explored further in the following manner:
2. Is this activity phase synchronous or coherent with the muscular activity or the activity in another brain region (measurement through a further deep electrode or via an epicordical or an EEG scalp electrode)?
3. What is the directionality of the interaction between the two measurement signals? Is the LFP (local field potential) of the deep electrode driven by the peripheral muscular activity or vice versa? In this case a distinction is to be made as to whether the deep region is a generator of the peripheral tremor or whether in the target area there is a proprioceptive feedback, that is a feedback from the function state, for example, the musculature or tendents which can be detected.
4. Aside from the exploration of the “spontaneous”, that is the signals recovered without stimulation, additionally the reaction of the measurement signals (for example the target regions and the musculature activity) to standardized tests stimuli can be objectionally explored and classified. In this case it is possible to test how on the one hand the target region and on the other hand the interaction between the target region and another brain region or between target regions and peripheral musculature activity can be present through the use of a standardized application of stimuli (for example short high frequency excitation (greater than 100 Hz), periodic pulses with a pulse frequency in the region of the tremor frequency). For classification or functional identification of the target region, the results can be compared advantageously with a reference databank online.

The device according to the invention is especially useful in the practice of medicine, and especially in neurology and psychiatry.

1. A device for localizing the target point of electrodes for brain stimulation, characterized in that it encompasses an isolation amplifier (1) with at least one electrode (2) and at least one sensor (3):

2. The device according to claim 1 characterized in that the sensor (3) is an EEG scalp electrode, an epicordical electrode, a deep electrode, a brain electrode, a peripheral electrode, a muscular electrode or a combination of at least two of these sensors (3).

3. The device according to claim 1 characterized in that the isolation amplifier (1) is connected to an evaluating unit (4).

4. The device according to claim 3 characterized in that the evaluating unit (4) comprises a univariate and bivariate or multivariate data processing.

5. The device according to claim 3 characterized in that it has means for visualizing the data.

6. The device according to claim 3 characterized in that it has a control unit (4, 4a) which enables the generation of stimuli through the electrode (2).

7. The device according to claim 3 characterized in that it effects the generation of stimuli through the electrode (2) through a coupling which has means for galvanic decoupling (5).

8. The device according to claim 7 characterized in that the galvanic decoupling means (5) encompasses an optical transmitter and an optical receiver which supplies signals to the electrode (2).

9. The device according to claim 3 characterized in that it has means for preventing overdriving of the isolating amplifier.

10. The device according to claim 9 characterized in that the means for preventing overdriving of the isolating amplifier (1) is a relay or a transistor or an electronic filter (9).

11. The device according to claim 1 characterized in that the electrode (2) and the sensor (3) are at least partly encompassed in the same component.

12. The use of the device according to claim 1 in the practice of medicine.
